(19) United States
(12) Patent Application Publication
(10) Pub. No.: US 2013/0115169 A1
(43) Pub. Date: May 9, 2013

(54) RED BLOOD CELL-MIMETIC PARTICLES AND METHODS FOR MAKING USE THEREOF

(71) Applicants: Joerg Lahann, Ann Arbor, MI (US); Samir Mitragotri, Santa Barbara, CA (US); Srijanani Bhaskar, Ann Arbor, MI (US); Nishit Doshi, Goleta, CA (US); Alisar A. Zahr, Acton, MA (US)

(72) Inventors: Joerg Lahann, Ann Arbor, MI (US); Samir Mitragotri, Santa Barbara, CA (US); Srijanani Bhaskar, Ann Arbor, MI (US); Nishit Doshi, Goleta, CA (US); Alisar A. Zahr, Acton, MA (US)

(73) Assignees: THE REGENTS OF THE UNIVERSITY OF CALIFORNIA, Oakland, CA (US); THE REGENTS OF THE UNIVERSITY OF MICHIGAN, Ann Arbor, MI (US)

(21) Appl. No.: 13/513,783

(22) PCT Filed: Dec. 3, 2010
(86) PCT No.: PCT/US10/58921

§ 371 Date: Jun. 4, 2012

Related U.S. Application Data
(60) Provisional application No. 61/266,927, filed on Dec. 4, 2009.

(51) Int. Cl. A61K 9/14

(52) U.S. Cl. CPC A61K 9/14
USPC 424/9.1; 424/400; 514/13.4; 424/78.35; 424/600; 424/700; 264/299; 264/484

(57) ABSTRACT

The present technology provides synthesized particles that mimic key structural and functional features of red blood cells. Such RBC-mimicking particles possess the ability to carry oxygen (and carbon dioxide) and flow through capillaries smaller than their own diameter. Further, such particles can also deliver drugs and imaging agents. These particles provide a new paradigm for the design of drug delivery and imaging carriers since they combine the functionality of natural RBCs with the broad applicability and versatility of synthetic drug delivery particles. Further, such particles can be used for detoxification and other biomedical applications.

### CROSS-REFERENCE TO RELATED APPLICATIONS

This application claims the benefit of U.S. Provisional Application No. 61/266,927, filed on Dec. 4, 2009. The entire disclosure of the above application is incorporated herein by reference.

### GOVERNMENT RIGHTS

This invention was made with government support under Contract Nos. HL080718 and RR017753-01 awarded by the National Institutes of Health. The government has certain rights in the invention.

### FIELD

The present disclosure relates to biomaterials, and more specifically red blood cell-mimetic particles, including methods for making and using such red blood cell-mimetic particles.

### BACKGROUND

This section provides background information related to the present disclosure which is not necessarily prior art.

Biomaterials form the basis of current and future biomedical technologies, like drug delivery, medical imaging and regenerative medicine. Several biomaterials, including polymeric nanoparticles and liposomes, can be used to design therapeutic carriers, such as nanoparticles, for applications in drug delivery. These biomaterials enhance the therapeutic benefit of drugs via sustained release, reduced side-effects and effective targeting. Various innovative strategies have been designed and implemented to optimize materials used for drug delivery. These include synthesis of new polymers to improve biocompatibility, fabrication of particles with various morphologies to control pharmacokinetics, modification of particle surface with polyethylene glycol to improve circulation and functionalization of particles with peptides and aptamers for targeted drug delivery.

While conventional synthetic drug delivery carriers have brought about numerous advances in drug delivery, they fail to match the sophistication exhibited by innate biological entities. For example, erythrocytes or red blood cells (RBCs) are the most ubiquitous cell type in the human blood and constitute highly specialized entities with unique shape, size, mechanical flexibility and material composition, all of which are optimized for extraordinary biological performance. In this context, red blood cells (RBCs), the most abundant cells in blood, represent a remarkably engineered biological entity designed for complex biological functionality including oxygen delivery. RBCs possess unique physical and chemical properties in terms of size, shape, mechanical flexibility and chemical composition, all of which are integral to their biological functions. It would be desirable to create synthetic particles that are capable of mimicking sophisticated RBC functionality in vitro or in vivo.

### SUMMARY

This section provides a general summary of the disclosure, and is not a comprehensive disclosure of its full scope or all of its features.

In various aspects, the present disclosure provides methods of making a red blood cell-mimetic particle. Such methods comprise providing a template particle comprising a polymer. The polymer of the template particle preferably comprises a pharmaceutically and/or cosmetically acceptable polymer. At least one bioactive agent is disposed on a surface of the template particle to form a stable bioactive layer. Then, the template particle is substantially removed with a treatment agent, so that the stable bioactive layer forms a particle having a substantially similar shape to a predetermined shape of a natural red blood cell.

In other aspects, the present inventive technology provides synthetic red blood cell particles comprising a stable bioactive layer comprising at least one bioactive agent. The synthetic red blood cell particles have a bi-concave discoid shape substantially similar to a predetermined shape of a natural red blood cell.

In yet other aspects, the present disclosure also provides a method of delivering a drug within an organism by introducing an inventive synthetic red blood cell particle (or a plurality of such particles) into the organism. In other variations, the present disclosure provides a method of delivering an active agent, such as a drug, to a target within an organism by introducing an inventive synthetic red blood cell particle (or a plurality of such particles) into the organism, where the bioactive agent includes an active agent and a targeting moiety for interacting with and delivering the synthetic red blood cell particle to the target.

In further variations, a method of diagnosing a disorder in an organism is provided by introducing one or more inventive synthetic red blood cell particles (or a plurality of such particles) into the organism, where the bioactive agent comprises a diagnostic imaging indicator agent. The present disclosure further provides a method of transporting oxygen in an organism by introducing the inventive synthetic red blood cell particles (or a plurality of such particles) into a circulatory system of the organism, where the bioactive agent comprises an oxygen-releasing component, such as hemoglobin that release oxygen. Likewise, a method of transporting carbon dioxide is provided in certain variations in an organism that includes introducing an inventive synthetic red blood cell particle (or a plurality of such particles) into a circulatory system of the organism, where the bioactive agent transports carbon dioxide.

Further, in other aspects the present disclosure provides for methods of detoxification for an organism having a toxin by introducing an inventive synthetic red blood cell particle (or a plurality of such particles) into the organism. The bioactive agent comprises an agent that interacts with one or more toxins to facilitate removal from the organism or neutralization of the toxin(s).

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations, and are not intended to limit the scope of the present disclosure.

Corresponding reference numerals indicate corresponding parts throughout the several views of the drawings.

FIGS. 1A-1C depict various aspects of the present teachings relating to methods of forming synthetic particles capable of RBC-mimicking. In FIG. 1A, RBC-shaped particles are prepared from hollow polystyrene (PS) template(s). In this aspect of the present teachings, complementary layers of proteins and polyelectrolytes are deposited by layer-by-layer (LbL) technique on a template surface followed by cross-linking of the layers to increase stability. The PS core of the template is dissolved to yield RBC-shaped particles, which can be loaded with active agents, like therapeutic and/or imaging agents, by way of non-limiting example. In FIG. 1B, another variation of the present teachings is shown, where biocompatible RBC-mimicking particles are prepared from PLGA template particles. First, PLGA spheres are formed via electrohydrodynamic jetting. Then, RBC-shaped templates are synthesized by incubating the spheres synthesized via electrohydrodynamic jetting in 2-propanol. A layer-by-layer coating is formed on a surface of the template, followed by protein cross-linking and dissolution of template core to yield biocompatible RBCs. FIG. 1C shows a general schematic of a particle or cell exhibiting a biconcave discoidal shape;

FIGS. 2A-2C show scanning electron microscope (SEM) micrographs of the RBC-mimicking particles fabricated according to the process depicted in FIG. 1A, which uses hollow PS template particles. FIG. 2A has bovine albumin serum (BSA) and poly(allyamine) hydrochloride (PAH) deposited on template particles by a layer-by-layer (LbL) application process, where the layers are cross-linked. Then, the particles are exposed to tetrahydrofuran (THF) to yield synthetic RBCs (sRBCs). The inset of FIG. 2A shows a close-up. FIG. 2B shows another embodiment, where hemoglobin (Hb) and poly(4-styrene sulfonate) (PSS) are applied to a hollow PS template according to the process of FIG. 1A by an LbL technique. In FIG. 2C, sRBCs are prepared by adsorption of Hb on template particles. (All scale bars in figures are 1 μm, inset 500 nm);

FIGS. 3A-3C show scanning electron microscope (SEM) micrographs of another aspect of the present teachings, where RBCs are formed using biocompatible polymers. In FIG. 3A, RBC-shaped polymeric templates (PLGA) are fabricated by electrohydrodynamic jetting. In FIG. 3B, biodegradable RBCs are prepared from PLGA template particles by LbL deposition of PAH/BSA and subsequent dissolution of the polymer template core. FIG. 3C shows a cross-linked mouse RBCs. The particles made in accordance with the present teachings are substantially similar to their natural counterpart(s). Insets show close-up images. (All scale bars 5 μm, insets 2 μm);

FIGS. 4A-4B show mechanical properties of synthetic red blood cells (sRBCs) made in accordance with the present teachings and measured using atomic force microscopy (AFM). FIG. 4A shows a comparison of elastic modulus of sRBCs with natural mouse RBCs and PLGA particles linked natural mouse RBC. The RBCs made according to (*p<0.001, n=5). FIG. 4B shows sRBCs (7±2 μm) made in accordance with the methods of the present disclosure flowing through glass capillary (5 μm inner diameter). The image also shows a particle outside the capillary (scale bar 5 μm);

FIGS. 5A-5C show exemplary biomedical applications for synthetic red blood cells (sRBCs) made in accordance with the methods of the present disclosure. FIG. 5A demonstrates oxygen carrying capacity of sRBCs based on a chemiluminescence reaction of luminol. Cross-linking and exposure to an organic solvent reduces the oxygen carrying capacity, but coating the sRBCs with uncrosslinked hemoglobin (Hb) increases the oxygen-binding capacity to levels comparable to mouse blood (S-RBC, t=0). Ninety percent of oxygen carrying capacity is retained after one week (S-RBC, t=1 wk). Bovine serum albumin (BSA)-coated particles are included as negative control (*p<0.01, n=3). FIG. 5B shows a controlled release of radiolabeled heparin from sRBCs made in accordance with the present teachings over a period of 10 days (n=5). FIG. 5C shows a transmission electron microscope (TEM) micrograph showing encapsulation of iron oxide nanoparticles having an average particle size of 30 nm in biodegradable synthetic RBC-shaped particles. The inset shows PLGA particles loaded with iron oxide prior to conversion into RBC-like templates (scale bars 1 μm);

FIGS. 6A-6C show elastic modulus of synthetic red blood cells (sRBCs) determined from force-indentation curves measured using atomic force microscopy (AFM). Specifically, measurement of elastic modulus of sRBCs formed in accordance with the present disclosure are depicted in FIG. 6A; elastic modulus of natural mouse RBCs in FIG. 6B; and force versus time loading-unloading cycle used for indentation in FIG. 6C;

FIGS. 7A-7B show biocompatible synthetic RBCs prepared in accordance with the present disclosure by depositing alternate layers of BSA/Hemoglobin (Hb) fabricated by a layer-by-layer self-assembly (LbL) technique where a shell is composed of alternate layers of BSA and Hb (scale bar 5 μm);

FIGS. 8A-8B show controlled release of Texas-Red conjugated Dextran (3 kDa: solid circles and 10 kDa: solid squares) from synthetic RBCs over a period of about 10 days. FIG. 8A shows a controlled release profile of dextran (3 kDa and 10 kDa) and FIG. 8B shows a confocal image of Texas-Red dextran loaded synthetic RBC particles (scale bar 2 μm);

FIG. 9 shows elliptical synthetic red blood cells formed in accordance with one embodiment of the present disclosure, where hollow polystyrene (PS) spheres (oblate ellipsoids using the film stretching technique described in Champion, J. & Mitragotri, S. (2006), “Role of target geometry in phagocytosis” Proceedings of the National Academy of Sciences 103(13), 4930-4934, incorporated herein by reference). These particles can be used as templates to synthesize ellipsoidal RBC shaped particles which mimic the RBCs in hereditary elliptocytosis disorder, as shown in FIG. 9;

FIGS. 10A-10C depict measurement of oxygen binding capacity via chemiluminescence of synthetic RBCs formed in accordance with the present disclosure (FIG. 10A); natural mouse blood (FIG. 10B); and bovine serum albumin (BSA)-coated particles (FIG. 10C) demonstrating via chemiluminescence the oxygen carrying capacity of synthetic RBCs prepared in accordance with the present disclosure. The particles are prepared by adding 100 μL of Luminol solution to hemoglobin (Hb) coated synthetic RBCs. The image clearly shows bright blue chemiluminescence in the 96 well plate indicating the oxygen carrying capacity of synthetic RBCs.

### DETAILED DESCRIPTION

Example embodiments will now be described more fully with reference to the accompanying drawings. Further areas of applicability will become apparent from the description provided herein. The description and specific examples in this summary are intended for purposes of illustration only and are not intended to limit the scope of the present disclosure.

Example embodiments are provided so that this disclosure will be thorough, and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms and that neither should be construed to limit the scope of the disclosure. In some example embodiments, well-known processes, well-known device structures, and well-known technologies are not described in detail.

The terminology used herein is for the purpose of describing particular example embodiments only and is not intended to be limiting. As used herein, the singular forms “a”, “an” and “the” may be intended to include the plural forms as well, unless the context clearly indicates otherwise. The terms “comprises,” “comprising,” “including,” and “having,” are inclusive and therefore specify the presence of stated features, integers, steps, operations, elements, and/or components, but do not preclude the presence or addition of one or more other features, integers, steps, operations, elements, components, and/or groups thereof. The method steps, processes, and operations described herein are not to be construed as necessarily requiring their performance in the particular order discussed or illustrated, unless specifically identified as an order of performance. It is also to be understood that additional or alternative steps may be employed.

When an element or layer is referred to as being “on”, “engaged to”, “connected to” or “coupled to” another element or layer, it may be directly on, engaged, connected or coupled to the other element or layer, or intervening elements or layers may be present. In contrast, when an element is referred to as being “directly on,” “directly engaged to”, “directly connected to” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

The present disclosure provides methods for synthesizing particles that mimic the key structural and functional features of erythrocytes or red blood cells (RBCs). Similar to their natural counterparts, RBC-mimetic particles described herein possess the ability to carry oxygen and flow through capillaries smaller than their own diameter. Further, such RBC-mimetic particles optionally encapsulate drugs and/or imaging agents. The particles formed in accordance with the present techniques provide a new paradigm for design of drug delivery and imaging carriers, by way of non-limiting example, since they combine the functionality of natural RBCs with the broad applicability and versatility of synthetic particles, enabling drug delivery for example.

In various aspects, the particles formed in accordance with the present teaching are synthetic carriers that mimic the key structural attributes of RBCs including size, shape and mechanical properties, yet offer engineering control and design capabilities of synthetic carriers. Such synthetic RBC-mimetic particles can be used in a variety of biomedical applications, including in vitro and in vivo applications. These particles provide a path to bridge the gap between synthetic materials and biological entities.

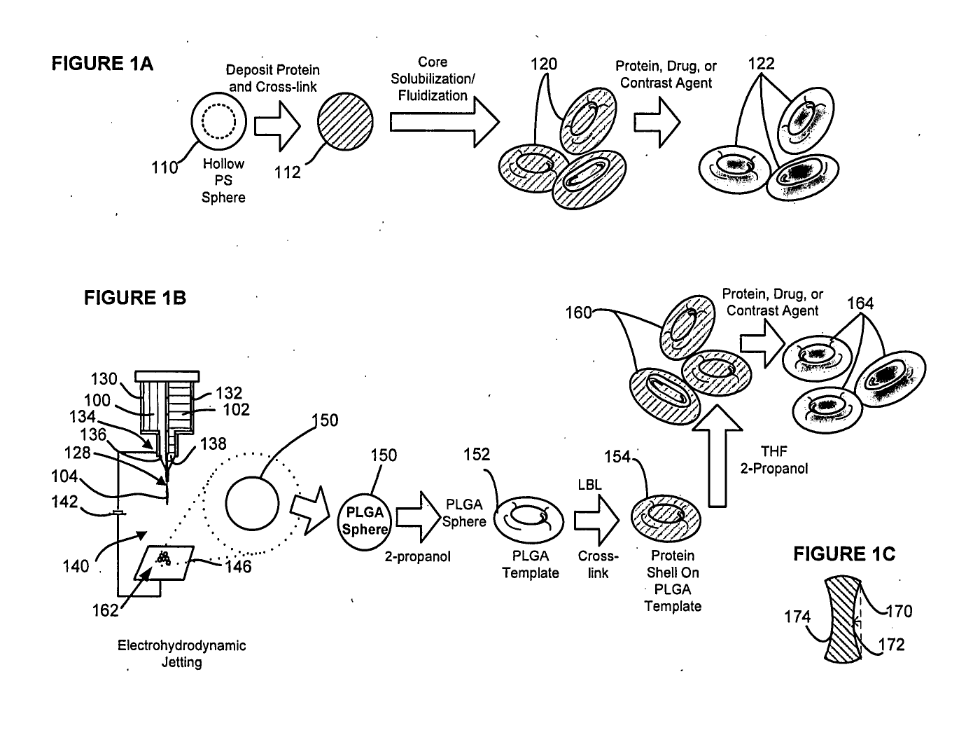

The structure of natural RBCs is characterized by several unique properties, including biconcave discoidal shape (see for example, FIG. 1C, having a disk shape with two generally concave central depressions 172, 174 (as compared to the outer peripheral region 170 of the disk) on either side of the disk). Natural RBCs have mechanical flexibility that so far have been unmatched by synthetic particles, which are typically spherical and stiff. Unique structural properties of RBCs allow them to routinely pass through ultrathin capillaries smaller than their own diameter and sinusoidal slits in the spleen. The biconcave discoidal shape also provides a favorable surface area to volume ratio and allows RBCs to undergo marked deformations while maintaining a constant surface area.

By way of background, the unique morphological properties of RBCs are achieved by a well-orchestrated series of biochemical events. RBCs originate as spherical reticulocytes, which make a transition into the biconcave shape during maturation over a period of 2-3 days. In nature, initial spherical reticulocytes, which have an elastic modulus of about 3 MPa undergo a 100-1000 fold reduction in elastic modulus and simultaneous change in shape to form discoidal RBCs.

In various aspects, the present disclosure pertains to methods for forming synthetic red blood cells. As appreciated by those of skill in the art, the present methods may also be used to make particles similar to other cells having predetermined shapes; however, in certain aspects, the present technology pertains to formation of particles mimicking the shape and function of red blood cells. For example, the present disclosure provides methods of making a red blood cell-mimetic particle that comprise providing a template particle comprising a polymer. In certain aspects, the polymer that forms the template particle is a pharmaceutically and/or cosmetically acceptable polymer, as are known by those of skill in the art. One example of a particularly suitable polymer is polystyrene (PS) or poly(lactide-co-glycolide polymer (PLGA), as will be discussed in further detail below.

In various aspects, a template particle has a shape that is substantially similar to a shape of a red blood cell, such as a healthy red blood cell or a malformed red blood cell causing a diseased condition in an organism, like a mammal or in particular in a human. In certain embodiments, the providing of the template particle further comprises incubating the template particle having a first shape in the presence of an agent that induces the template particle to have a second shape distinct from the first shape. Thus, in certain variations, the first shape may be a sphere or an ellipsoid, and the second shape (after incubating or treatment) is a biconcave discoid shape mimicking that of a red blood cell. In yet other embodiments, the providing may comprise forming the template particle by electrohydrodynamic jetting that comprises jetting a liquid stream passing through an electric field generated by electrodes sufficient to form a cone jet. In certain aspects, the template particles formed by electrohydrodynamic jetting may have a substantially spherical, a substantially ellipsoidal (including ovular), and a substantially disk/discoid shape. Where the electrohydrodynamic formation process is used to form a substantially spherical or substantially elliptical shape, it can be incubated in the process step described just above in the presence of an agent that forms a collapsed shape, such as a biconcave discoid shaped particle. In other aspects, the template particle formed via electrohydrodynamic jetting has a disk/discoid shape, which optionally may have a biconcave discoid shape and therefore may not require any further treatment or incubation to create the desired shape in the template particle.

In certain variations, the method further includes disposing at least one bioactive agent on a surface of the template particle to form a stable bioactive layer. The terms “biofunctional agent” and “bioactive agent” and “active ingredients” are used interchangeably herein. In certain aspects, one or more layers of the sRBC particles comprise a “bioactive” agent, which refers to a material or chemical substance, such as a small molecule, active ingredient, macromolecule, ligand, metal ion, or the like, that is bioactive and causes an observable change in the structure, function, optical function, or composition of a target cell, when such a target cell is exposed to such a material or substance. Non-limiting examples of observable cellular changes include increased or decreased expression of one or more mRNAs, DNA, or other nucleotides, increased or decreased expression of one or more proteins, phosphorylation of a protein or other cell component, inhibition or activation of an enzyme, inhibition or activation of binding between members of a binding pair, an increased or decreased rate of synthesis of a metabolite, increased or decreased generation of immune system cells, hormones, growth factors, or other intercellular mediators and signaling agents, increased or decreased cell proliferation, enhanced cellular growth, such as germline or somatic cell growth, changes in optical properties, and the like. In various aspects of the present disclosure, the bioactive agent promotes cellular development affecting cell shape, size, proliferation, growth, death, motility, state of differentiation, interaction with other cells, interaction with extracellular materials, or transcriptional, translational, or metabolic profile. In certain variations, the sRBC particles of the present disclosure are capable of delivering active ingredients to a target, which in some embodiments is to cells, tissue or to an organ of an organism. In yet other variations, the sRBC particles are capable of removing undesirable components from an environment, such as toxins, pathogens, target organisms or cells, and the like.

In certain variations, at least one layer of the synthetic particle comprises a bioactive ingredient that is a pharmaceutically active ingredient, which refers to a material or combination of materials that are used with mammals or other organisms having acceptable toxicological properties for beneficial use with such an animal. By way of non-limiting example, the bioactive/biofunctional agent included in one or more layers of a synthetic particle can be a therapeutic drug that operates locally or systemically (non-localized) and may treat, prevent, or diagnose a wide variety of conditions or ailments. In certain aspects, such active ingredients can be provided in one or more layers the sRBC particles to provide benefits in vivo. It should be appreciated that any agent discussed in the context of the present disclosure may have efficacy in several categories of an active agent and a discussion or listing of such an active agent under a given category is not exclusive or limiting of the active agent's utility.

Suitable non-limiting bioactive agents that are active ingredients may include those selected from the group consisting of: a therapeutic active ingredient, a systemic active ingredient, a chemotherapy active ingredient, a localized active ingredient, a nutritional active ingredient, a diagnostic imaging indicator agent, and combinations thereof. Such a bioactive agent may be combined with other bioactive agents or other structural materials that form the bioactive layer or may be applied independently to form the layer. After the bioactive agent is located on the surface of template particle, the bioactive agent can optionally be cross-linked to form a stable bioactive layer on the surface of the template particle. A wide variety of bioactive agents known in the art are contemplated, as is use of a plurality of distinct bioactive agents.

Thus, such an active ingredient is a compound or composition that diagnoses, prevents, or treats a physiological or psychological disorder or condition, or can provide a therapeutic, regenerative, cosmetic or aesthetic benefit in an organism, such as an animal, e.g., a mammal like a human. In certain aspects, a pharmaceutically active ingredient prevents or treats a disease, disorder, or condition of hard or soft tissue in an organism, such as a mammal. A biofunctional agent disposed on the bioactive layer as a bioactive agent can provide biological activity, aesthetic, sensory, cosmetic, cleansing/detoxifying, or nutritional benefits, by way of non-limiting example, or be included to target a particular region in the mammal, such as organs, tissues, medical implants or devices, skeletal system, hair, skin, mouth, eyes, circulatory system, and the like. Bioactive agents also encompass therapeutic agents, such as pharmaceutically active agents, like drugs, and also genetic materials and biological materials.

Thus, in certain variations, the biofunctional agent is a pharmaceutically active composition. Pharmaceutically active compositions include drug and therapeutic compositions, oral care compositions, nutritional compositions, personal care compositions, cosmetic compositions, diagnostic compositions, and the like. In various aspects, the synthetic RBC particle may be used in a wide variety of different biological (either in vitro or in vivo) applications and may have other biofunctional agents, and are not limited those described herein. However, the present disclosure contemplates synthetic RBC particles comprising one or more biofunctional agents that provide a diagnostic, therapeutic, prophylactic, cleansing/detoxifying, nutritional, cosmetic, sensory, and/or aesthetic benefit to an organism, such as a mammal like a human. In certain aspects, the synthetic RBC particles optionally comprise one or more bioactive agents, which optionally may be provided in a biocompatible composition in the respective layers of the sRBC.

Thus, biofunctional/active ingredients may be used to repair or regenerate cells of an organ or tissue; treat or prevent a disease, such as an infectious disease (a bacterial, viral, or fungal infection) or a degenerative disease (Alzheimer's, amyotrophic lateral sclerosis (ALS)). For example, active ingredients may treat an auto-immune disorder (e.g., rheumatoid arthritis, systemic lupus erythematosus (SLE), inflammatory bowel disease (IBD)), allergies, asthma, osteoarthritis, osteoporosis, cancer, diabetes, arteriosclerosis and cardiovascular disease, stroke, seizures, psychological disorders, pain, acne, caries, gingivitis, periodontitis, an H₂ antagonist, human immunodeficiency, infections, and the like. Particularly suitable bioactive agents for synthetic RBC particles include agents used to transport molecules inside an organism (e.g., transport oxygen, carbon dioxide, cell signaling compounds, and the like), minimize an organism's immune response to foreign matter (e.g., to reduce host rejection), to reduce thrombosis and clotting, to reduce pain, infection, and inflammation, to promote adhesion of certain target cells, to promote healing, cellular repair, and growth, and to promote tissue differentiation and proliferation, by way of non-limiting example.

The description of suitable biofunctional agents/active ingredients is merely exemplary and should not be considered as limiting as to the scope of biofunctional active ingredients which can be introduced into the synthetic RBC particle according to the present disclosure, as all suitable biofunctional agents and/or active ingredients known to those of skill in the art for these various types of compositions are contemplated. Furthermore, a biofunctional agent/active ingredient may have various functionalities and thus, can be listed in an exemplary class below; however, may be categorized in several different classes of active ingredients. Various suitable active ingredients are disclosed in Merck Index, An Encyclopedia of Chemicals, Drugs, and Biologicals, Thirteenth Edition (2001) by Merck Research Laboratories and the International Cosmetic Ingredient Dictionary and Handbook, Tenth Ed., 2004 by Cosmetic Toiletry and Fragrance Association, and U.S. Pat. Nos. 6,589,562, 6,825,161, 6,063,365, and 6,491,902, all to Shefer et al, each incorporated herein by reference. All references cited or described herein are hereby expressly incorporated by reference in their respective entireties.

More specifically, suitable bioactive agents include by way of non-limiting example, hemoglobin, bovine serum albumin (BSA), poly(allylamine hydrochloride) (PAH), hemoglobin (Hb), transcriptional activators; translational promoters; anti-proliferative agents; growth factors; growth factor receptors; growth hormones; anti-rejection drugs; anti-thrombotic agents; anti-coagulants; stem cell or gene therapies; antioxidants; free radical scavengers; nutrients; co-enzymes; ligands; cell adhesion peptides; peptides; proteins; nucleic acids; DNA; RNA; polysaccharides; sugars; nutrients; hormones; antibodies; immunomodulating agents; growth factor inhibitors; growth factor receptor antagonists; transcriptional repressors; translational repressors; replication inhibitors; inhibitory antibodies; cytotoxin; hormonal agonists; hormonal antagonists; inhibitors of hormone biosynthesis and processing; antigestagens; antiandrogens; anti-inflammatory agents; non-steroidal anti-inflammatory agents (NSAIDs); COX-I and II inhibitors; antimicrobial agents; antiviral agents; antifungal agents; antibiotics; anti-proliferative agents; antineoplastic/antiproliferative/anti-miotic agents; anesthetic, analgesic or pain-killing agents; antipyretic agents, prostaglandin inhibitors; platelet inhibitors; DNA de-methylating agents; cholesterol-lowering agents; vasodilating agents; endogenous vasoactive interference agents; angiogenic substances; cardiac failure active ingredients; targeting toxin agents; aptamers; quantum dots; nano-materials; nano-crystals; and combinations thereof. In certain variations, particularly preferred bioactive agents include bovine serum albumin (BSA), poly(allylamine hydrochloride) (PAH), hemoglobin (Hb), and combinations thereof.

A bioactive agent may be disposed on an exposed surface of the synthetic particle (for example, on one or more bioactive layers) and may comprise a biofunctional agent or moiety. In certain aspects, biofunctional agents, such as ligands, peptides (particularly cell adhesion peptides), cell adhesion molecules, proteins, nucleic acids, growth factors, hormones, antibodies, sugars, saccharides, nutrients, and the like can be included in the bioactive layer, deposited on or reacted with components in the bioactive layer at a surface.

In certain aspects, the moiety may be provided to interact with the surrounding environment (for example, to avoid detection by an immune system, provide optical properties to synthetic RBC particle, provide binding to a biological or non-biological target, such as cells or tissue or a medical device). In some aspects, the moiety is a binding moiety that provides the ability for the sRBC particles to bind with a target. In certain aspects, the target may be a cell of an organism, such as germline or somatic cells, protein, enzyme, immune system cells, or other circulating cells or substances associated with an organism or an animal.

Genetic materials encompass without limitation nucleotides or nucleic acids intended to be inserted into a human body, including viral vectors and non-viral vectors. Such bioactive agents optionally include cytokines, hormones, naturally occurring growth factors, proteins, peptides, peptoids, and small molecules identified by selection from chemical libraries, by way of non-limiting example.

Other suitable bioactive agents, which may optionally be surface bound moieties on one or more layers of the synthetic RBC particle, can include a growth factor. Many transforming growth factors (TGF-β super family) can be used for a wide range of therapeutic treatments and applications, which in particular, pertain to promotion of cell proliferation and tissue formation, including wound healing, tissue reproduction, and tissue regeneration. Furthermore, the synthetic RBC particle optionally comprises bioactive agents that inhibit growth or response of certain targeted tissues, for example, cancer or immune system cells. In certain aspects, the synthetic RBC particles have one layer comprising a bioactive agent to promote growth, proliferation, differentiation and/or repair of certain target cells, while another distinct bioactive agent may inhibit growth of distinct target cells. For example, a synthetic RBC particle optionally includes growth factors, growth factor receptors, transcriptional activators, and translational promoters for promoting cell growth (e.g., in the circulatory system or in organs, such as the heart, lungs, liver and the like) and may further optionally include cell growth inhibitors such as antiproliferative agents, growth factor inhibitors, growth factor receptor antagonists, transcriptional repressors, translational repressors, replication inhibitors, inhibitory antibodies, antibodies directed against growth factors, bioactive molecules consisting of a growth factor and a cytotoxin, bioactive molecules consisting of an antibody and a cytotoxin, and the like.

Further, various peptides are well known in the art for binding to cells in the brain, kidneys, lungs, skin, pancreas, intestine, uterus, adrenal gland, and prostate, including those described in Pasqualini et al, “Searching for a molecular address in the brain,” Mol Psychiatry. 1(6) (1996) pp. 421-2 and Rajotte, et al., “Molecular heterogeneity of the vascular endothelium revealed by in vivo phage display,” J Clin. Invest. 102(2) (1998) pp. 430-437, incorporated by reference herein. Other cell adhesion peptides, growth factors, antibodies, sugars, nucleotides, DNA, and the like known in the tissue and bioengineering arts may also be suitable moieties or ligands for the surface(s) of respective layers of the synthetic RBC particles.

Proteins, such as heat shock protein HSP70 for dendritic cells and folic acid to target cancer cells can be suitable ligand moieties for the surface of a layer of a synthetic RBC particle. Other suitable surface moieties include polysaccharides or sugars, such as silyilic acid for targeting leucocytes, targeting toxins such as saporin, antibodies, including CD 2, CD 3, CD 28, T-cells, and other suitable antibodies are listed in a Table at http://www.researchd.com/rdicdabs/cdindex.htm (Jun. 14, 2007), incorporated by reference. Other suitable binding moieties include aptamers, which are small oligonucleotides that specifically bind to certain target molecules, for example, Aptamer O-7 which binds to osteoblasts; Aptamer A-10 which binds to prostate cancer cells; and Aptamer TTA1, which binds to breast cancer cells. Other binding biological binding moieties suitable for tissue engineering or cell cultures known or to be developed in the art are contemplated by the present disclosure.

As noted above, such bioactive agents are optionally included throughout one or more layers of the synthetic RBC particles or may be provided only on the surface of an exposed layer (as a surface bound moiety), as will be described in greater detail below.

Further, the synthetic RBC particles may include immunotherapeutic agents, such as antibodies and immunomodulators, which may inhibit growth of certain target cells, which include by way of non-limiting example, HERCEPTIN™ (trastuzumab, humanized IgG1 antibody for metastatic breast cancer); RITUXAN™ (Rituximab, chimeric IgG1 antibody for NHL); PANOREX™ (17-1A monoclonal antibody), BEC2 (anti-idiotypic antibody), IMC-C225 (monoclonal antibody); VITAXIN™ (monoclonal antibody); CAMPATHUH™ (DNA-derived humanized monoclonal antibody), 5G1.1 (humanized IgG for treatment of rheumatoid arthritis (RA), systemic lupus erythematosus (SLE), nephritis); 5G1.1-SC (humanized ScFv antibody for cardiopulmonary bypass, infarction, angioplasty and other cardiac procedures); ABX-CBL (humanized antibody for graft-versus-host disease (GvHD)); ABX-CBL (murine CD147 antibody for allograft rejection); ABX-IL8 (humanized IL-8 antibody for psoriasis); AD-159 (humanized antibody for human immunodeficiency virus (HIV)); AD-439 (humanized antibody for HIV); ANTEGREN™ (humanized IgG antibody for multiple sclerosis); Anti-CD11a (humanized IgG1 antibody for psoriasis); Anti-CD18 (humanized Fab′2 antibody for myocardial infarction); Anti-LFA1 (murine Fab′2 antibody for allograft rejection); Anti-VEGF (humanized IgG1 antibody for cancer); ANTOVA™ (humanized IgG antibody allograft rejection); BEC2 (murine IgG antibody for lung cancer); BIRR-1 (murine IgG2a antibody for stroke); BTI-322 (Rat IgG antibody GvHD); C225 (chimeric IgG antibody for head and neck cancers); CAT-152 (humanized antibody glaucoma); CDP571 (humanized IgG4 antibody for Crohn's disease); CDP850 (humanized antibody for psoriasis); CORSEVIN M™ (chimeric antibody as an anticoagulant); D2E7 (humanized antibody for RA); Hu23F2G (humanized IgG antibody for stroke and MS); ICM3 (humanized antibody for Psoriasis); IDEC-114™ (primatized antibody for psoriasis); IDEC-131™ (humanized antibody for SLE, multiple sclerosis (MS)); IDEC-151™ (primatized IgG1 for RA); IDEC-152™ (primatized antibody for asthma and allergic reactions); INFLIXIMAB™ (chimeric IgG1 antibody for RA, Crohn's disease); LDP-01 (humanized IgG antibody for stroke, allograft rejection); LDP-02 (humanized antibody for ulcerative colitis); LDP-03/CAMPTATH 1H™ (humanized IgG1 antibody for chronic lymphocytic leukemia (CLL)); Lym-1 (chimeric antibody for non-Hodgkin's lymphoma (NHL)); LYMPHOCIDE™ (humanized antibody for NHL); MAK-195F (murine Fab′2 antibody for toxic shock); MDX-33 (human antibody for autoimmune haematogical disorders); MDX-CD4 (human IgG antibody for RA); MEDI-500 (murine IgM antibody for treating GvHD); MEDI-507 (humanized antibody for psoriasis and GvHD); OKT4A (humanized IgG antibody for allograft rejection); ORTHOCLONE™ (humanized IgG antibody for autoimmune disease); ORTHOCLONE™/anti-CD3 (murine mIgG2a antibody for allograft rejection); OSTAVIR™ (human antibody for Hepatitis B); OVAREX™ (murine antibody for ovarian cancer); PANOREX 17-1A™ (murine IgG2a antibody for colorectal cancer); PRO542 (humanized antibody for HIV); PROTOVIR™ (humanized IgG1 antibody for cytomegalovirus infection (CMV)); REPPRO/ABCIXIMAB™ (chimeric Fab antibody for complications from coronary angioplasty); rhuMab-E25 (humanized IgG1 antibody for asthma and allergies); SB-240563 (humanized antibody for asthma and allergies); SB-240683 (humanized antibody for asthma and allergies; SCH55700 (humanized antibody for asthma and allergies); SIMULECT™ (chimeric IgG1 antibody for allograft rejection); SMART a-CD3™ (humanized IgG antibody for autoimmune disease, allograft rejection, and psoriasis); SMART M195™ (humanized IgG antibody for Acute Myeloid Leukemia (AML)); SMART I D10™ (antibody for NHL); SYNAGIS™ (humanized IgG1 antibody for RSV); VITAXIN™ (humanized antibody for Sarcoma); and ZENAPAX™ (humanized IgG1 antibody for allograft rejection), and combinations thereof.

In certain other embodiments, the synthetic RBC particles may further comprise a hormonal treatment agent, such as hormonal agonists, hormonal antagonists (e.g., flutamide, tamoxifen, leuprolide acetate (LUPRON™), LH-RH antagonists), inhibitors of hormone biosynthesis and processing, steroids (e.g., dexamethasone, retinoids, betamethasone, cortisol, cortisone, prednisone, dehydrotestosterone, glucocorticoids, mineralocorticoids, estrogen, testosterone, progestins), antigestagens (e.g., mifepristone, onapristone), antiandrogens (e.g., cyproterone acetate), and combinations thereof, by way of non-limiting example.

Thus, in certain aspects, the synthetic RBC particles of the present disclosure optionally comprise one or more bioactive agents selected from: anti-rejection drugs (such as cyclosporine), anti-inflammatory agents, non-steroidal anti-inflammatory agents (NSAIDs), COX-I and II inhibitors, antioxidants, antimicrobial agents, including antiviral, antifungal, antibiotics and the like, and combinations and equivalents thereof. For example, useful anti-inflammatory agents include steroids, such as glucocorticoids, betamethasone, dexamethasone, prednisolone, corticosterone, budesonide, estrogen, sulfasalazine, and mesalamine, while indomethacin, ibuprofen, naproxen, and the like are suitable NSAIDs for incorporation into one or more layers of the synthetic RBC particles. Suitable antibiotic agents, include penicillin, cefoxitin, oxacillin, tobranycin, rapamycin, by way of non-limiting example.

Other bioactive agent materials also include non-genetic therapeutic agents, such as: anti-thrombogenic agents such as heparin, heparin derivatives, urokinase, and PPack (dextrophenylalanine proline arginine chloromethylketone); anti-proliferative agents such as enoxaprin, angiopeptin, or monoclonal antibodies capable of blocking smooth muscle cell proliferation, hirudin, and acetylsalicylic acid, amlodipine and doxazosin; antineoplastic/antiproliferative/anti-miotic agents such as paclitaxel, 5-fluorouracil, cisplatin, vinblastine, cladribine, vincristine, epothilones, methotrexate, azathioprine, adriamycin and mutamycin; endostatin, angiostatin and thymidine kinase inhibitors, Taxol™ and its analogs or derivatives; anesthetic or pain-killing agents such as lidocaine, bupivacaine, and ropivacaine; anti-coagulants such as D-Phe-Pro-Arg chloromethyl keton, RGD peptide-containing compound, heparin, anti-thrombin compounds, anti-thrombin antibodies, platelet receptor antagonists, anti-platelet receptor antibodies, aspirin (aspirin is also classified as an analgesic, antipyretic and anti-inflammatory drug), dipyridamole, protamine, hirudin, prostaglandin inhibitors, platelet inhibitors and tick anti-platelet peptides; DNA de-methylating drugs such as 5-azacytidine, (also categorized as a RNA or DNA metabolite that inhibits cell growth and induce apoptosis in certain cancer cells); cholesterol-lowering agents; vaso-dilating agents; and agents that interfere with endogenous vasoactive mechanisms; anti-oxidants, such as probucol; angiogenic substances, such as acidic and basic fibroblast growth factors, estrogen including estradiol (E2), estriol (E3) and 17-Beta Estradiol; drugs for heart failure, such as digoxin, beta-blockers, angiotensin-converting enzyme (ACE) inhibitors including captopril, enalopril, and statins and related compounds.

In some aspects, it may be desirable to avoid detection of the synthetic RBC particles by the animal's immune system, for example, to prevent removal or an immune system rejection response from the organism, like a human body, by macrophages and the like. The present disclosure contemplates various methods to prevent an animal's immune system from identifying the synthetic RBC particles and mounting an immune system response. In addition to the immunomodulator agents discussed above, another method to avoid immune response can be to provide moieties on the surface of at least one layer that is a “cloaking agent,” which prevents the animal's immune system from recognizing a foreign body. Examples of such moieties include modified carbohydrates, such as sialic acid, dextran, pullulan, or glycolipids, hyalluronic acid, chitosan, polyethylene glycols, and combinations thereof. Other examples of immune system cloaking agents known in the art or to be discovered are further contemplated.

Certain suitable bioactive active ingredients (e.g., pharmaceutically active ingredients or drugs, known to those of skill in the art) include, but are not limited to, low-molecular weight molecules, quantum dots, natural and artificial macromolecules, such as proteins, sugars, peptides, DNA, RNA, and the like, natural polymers, dyes and colorants, inorganic ingredients including nano-materials, and nano-crystals, fragrances, and mixtures thereof.

Additional ingredients that can be used in the synthetic red blood cells are not necessarily bioactive, but are used for diagnostic purposes, such as in various diagnostic medical imaging procedures (for example, radiographic imaging (x-ray), fluorescence spectroscopy, Forster/fluorescent resonance energy-transfer (FRET), computed tomography (CT scan), magnetic resonance imaging (MRI), positron emission tomography (PET), other nuclear imaging, and the like). Diagnostic ingredients for use with diagnostic imaging include contrast agents, such as iron oxide, or barium sulfate for use with MRI, for example, or fluorescein isothiocyanate (FITC).

In yet other aspects, the synthetic RBC particle optionally comprises a magnetic material or other component that is responsive to an external force field (for example, a charged surface) in one or more layers, so that in the presence of the force field (e.g., magnetic field), the synthetic RBC particles can be collected from an assay or sample, for example, in a laboratory or in vitro test.

Further, the synthetic RBC particles of the present disclosure may have a cleansing or detoxifying function in vitro or in vivo, to remove, destroy, or eliminate undesirable compounds, moieties, or cells. For example, the synthetic RBC particle optionally comprises a material capable of binding or otherwise removing a toxin (e.g., endogenous and exogenous toxins) or an endotoxin. In certain variations, the present disclosure provides a method of detoxification for an organism having a toxin that includes introducing a synthetic red blood cell particle into the organism, wherein the bioactive agent comprises an agent that interacts with said toxin to facilitate removal from the organism or neutralization of said toxin, as are known to those of skill in the art.

Endogenous toxins arise from a great many pathologic bodily processes. “Endotoxins” generally include bacterial cell wall lipopolysaccharides or lipooligosaccharides that elicit an inflammatory response. As used here, endotoxins further includes precursors and/or intermediaries on the biochemical pathway leading to endotoxic shock. During sepsis and septic shock, inflammatory mediators (IM) are excessively produced. For example, at a site of local tissue injury or infection, IMs serve an important vital immune function of removal and healing of injured or dead tissue, or resisting or destroying infecting organisms. When IMs become excessive and enter the general circulation, they may become toxic to the body causing a systemic inflammatory response syndrome, with potential complicating multi-organ dysfunction syndrome and multi-organ system failure.

Further, other diseases, such as rheumatoid arthritis, multiple sclerosis, lupus (SLE), graft versus host disease and similar conditions, are similarly caused by excess circulating IMs. These conditions generally result from an autoimmune process in which IMs, either physiologic or pathologic, are dysfunctionally produced and are therefore potentially toxic in any amount, or produced in dysfunctionally large and toxic quantities. Sepsis-septic shock and the autoimmune diseases, each resulting from dysfunctional and/or dysfunctionally abundant IMs, may be referred to in aggregate as inflammatory mediator related diseases.

Liver failure is a complex disorder with an intricate pathophysiology and diverse effects on many vital organs. It can be characterized by the accumulation in the body of many toxins that arise from metabolic processes, which under normal conditions, are quickly detoxified and eliminated by the liver, but which, in liver failure accumulate in the body. The pathologic effects of liver failure include the accumulation of various liver failure toxic substances, which can have a lethal effect on the animal.

Further, the synthetic RBC particles may include a compound capable of cleansing or inactivating exogenous toxin exposures, occurring through accidental and intentional toxin ingestions and environmental (industrial, agricultural, and the like) toxin exposures. Thus, the synthetic RBC particle optionally includes a bioactive agent that is targeted to remove, bind with, or inactivate a preselected toxin, whether endogenous or exogenous in nature.

Any of the above described biofunctional or bioactive active materials may be disposed on a surface of one or more layers of the sRBC particles or may be distributed throughout (e.g., homogeneously mixed) the material forming the layer (and thus, may be exposed at the surface, as well). For example, disposing a bioactive agent on the surface of a template to form such a bioactive layer can be conducted via a layer-by-layer (LbL) self assembly technique that electrostatically deposits cationic and anionic molecules or polymers on a surface of the template particle.

In certain preferred aspects, a suitable bioactive agent is selected from the group consisting of: bovine serum albumin (BSA), poly(allylamine hydrochloride) (PAH), hemoglobin (Hb), and combinations thereof. By way of non-limiting example, BSA is a polyanion and PAH is a polycation which can be respectively disposed on the surface of the template particle via LBL in sequential fashion, respectively (e.g., BSA layer, PAH layer, followed by BSA layer, and so forth). The present teachings also contemplate depositing other bioactive agents or other materials combined with the bioactive agents onto the template particles (portions of which may remain in the core region after the solubilization or fluidization collapsing process).

Suitable non-limiting polymers for use in the synthetic RBC particle template particles (e.g., in Fluid A designated 100 or Fluid B designated 102 of FIG. 1B) include a biodegradable polymer such as poly(lactide-co-glycolide polymer (PLGA), a polylactic acid, polycaprolactone, polyglycolic acid, copolymers, and derivatives thereof. As noted above, PLGA and polystyrene are particularly preferred materials for forming the template particle.

For the bioactive layers (to form the shell) of the synthetic RBC particle, in certain variations, preferred bioactive agents include bovine serum albumin (BSA), poly(allylamine hydrochloride) (PAH), hemoglobin (Hb), and combinations thereof. Other polyelectrolytes (or charged components) may likewise be used in such layer by layer assembly techniques, such as a polyelectrolyte selected from the group consisting of: polyacrylic acid (PAA), poly(acrylamide acrylic acid (PAAm), and/or poly(acryl amide-co-acrylic acid) (PAAm-AA), sodium polystyrene sulfonate (PSS), copolymers, and combinations thereof. In certain variations, other suitable biocompatible polymers include polyoxyethylene glycol or polyethylene glycol (PEG), polyethylene imine (PEI), polyvinylpyrrolidone (PVP), and mixtures thereof. Other polymers discussed herein include those well known to those of skill in the art to be used in cell cultures, implants, regenerative, therapeutic, and pharmaceutical compositions.

Suitable water-soluble and/or hydrophilic polymers, which are biocompatible, include cellulose ether polymers, including those selected from the group consisting of hydroxyl alkyl cellulose, including hydroxypropyl methyl cellulose (HPMC), hydroxypropyl cellulose (HPC), hydroxyethyl cellulose (HEC), methyl cellulose (MC), carboxymethyl cellulose (CMC), and mixtures thereof. Other polymers among those useful herein include polyvinylpyrrolidone (PVP), vinyl acetate, polyvinylpyrrolidone-vinyl acetate copolymers, polyvinyl alcohol (PVA), acrylates and polyacrylic acid (PAA), including polyacrylate polymer, vinylcaprolactam/sodium acrylate polymers, methacrylates, poly(acryl amide-co-acrylic acid) (PAAm-co-AA), vinyl acetate and crotonic acid copolymers, polyacrylamide, polyethylene phosphonate, polybutene phosphonate, polystyrene, polyvinylphosphonates, polyalkylenes, and carboxy vinyl polymer. The multiphasic fiber compositions may comprise derivatives, copolymers, and further combinations of such polymers, as well.

Other polymers or water-soluble fillers among those useful herein include, without limitation, sodium alginate, carrageenan, xanthan gum, gum acacia, Arabic gum, guar gum, pullulan, agar, chitin, chitosan, pectin, karaya gum, locust bean gum, various polysaccharides; starches such as maltodextrin, amylose, corn starch, potato starch, rice starch, tapioca starch, pea starch, sweet potato starch, barley starch, wheat starch, modified starch (e.g., hydroxypropylated high amylose starch), dextrin, levan, elsinan and gluten; and proteins such as collagen, whey protein isolate, casein, milk protein, soy protein, keratin, and gelatin.

Further, non-limiting examples of water insoluble or hydrophobic polymers include cellulose acetate, cellulose nitrate, ethylene-vinyl acetate copolymers, vinyl acetate homopolymer, ethyl cellulose, butyl cellulose, isopropyl cellulose, shellac, hydrophobic silicone polymer (e.g., dimethylsilicone), polymethyl methacrylate (PMMA), cellulose acetate phthalate and natural or synthetic rubber; siloxanes, such as polydimethylsiloxane (PMDS), polymers insoluble in organic solvents, such as cellulose, polyethylene, polypropylene, polyesters, polyurethane and nylon, including copolymers, derivatives, and combinations thereof. The polymers may be crosslinked after formation by application of heat, actinic radiation or other methods of curing and treating polymers known to those of skill in the art. Additionally, in certain aspects, other synthetic and natural biocompatible polymers known or to be discovered in the art are contemplated by alternate variations of the present disclosure.

Furthermore, at least one layer of the synthetic RBC particle can be designed to have one or more of the following properties based upon material selection: hydrophobic, positively-charged (cationic), negatively-charged (anionic), polyethylene glycol (PEG)-ylated, covered with a zwitterion, hydrophobic, superhydrophobic (for example having with water contact angles in excess of 150°), hydrophilic, superhydrophilic (for example, where the water contact angle is near or at 0°), olephobic/lipophobic, olephilic/lipophilic, and/or nanostructured, among others.

In other aspects, one or more polymers or materials used within a layer may be functionalized to subsequently undergo reaction with various moieties or substances after formation of the synthetic RBC particle, to provide desired surface properties or to contain various moieties presented on the layer surface (e.g., for surface patterning), as recognized by those of skill in the art.

Other conventional biocompatible materials can be used to form the materials of bioactive layers or template particles, including solvents, plasticizers, cross-linking agents, surface active agents, fillers, bulking, or viscosity modifying agents, pH modifiers, pH buffers, antioxidants, impurities, UV stabilizers, and where appropriate, flavoring, or fragrance substances.

In certain aspects, the synthetic red blood cells of the present disclosure deliver active ingredients to a target, which in some embodiments is to cells, tissue or to an organ of an organism. A bioactive agent can be incorporated into a synthetic red blood cell for enhanced drug targeting, or enhanced efficacy of active ingredient via improved delivery to a target site in an organism. So-called “drug targeting” modifies the pharmacokinetics and biodistribution of active ingredients to provide the potential for increased efficacy, while minimizing intrinsic toxicity. In certain variations, a targeting moiety may be incorporated as a bioactive agent into the synthetic blood cell having an active ingredient like a drug. The targeting moiety incorporated into a synthetic red blood cell is capable of reacting with a particular type of cell or other target to deliver a drug to selective target cells.

In various aspects, after deposition of the bioactive layer onto the template to form the bioactive layer with optional cross-linking, the methods of the present disclosure remove the template particle forming the core region (now having a stable bioactive layer disposed on its exterior) with a treatment agent so that the stable bioactive layer forms a particle having a substantially similar shape to a predetermined shape of a natural red blood cell. In certain aspects, the treatment agent comprises 2-propanol, tetrahydrofuran, or combinations thereof. In certain variations, the treatment agent comprises a mixture of 2-propanol and tetrahydrofuran. In certain aspects, a ratio of 2-propanol to tetrahydrofuran is about 1:2 on a weight basis, particularly when the template particle comprises PLGA as a polymer.

Thus, in various aspects, the present disclosure provides a synthetic red blood cell particle comprising a stable bioactive layer comprising at least one bioactive agent and having a bi-concave discoid shape substantially similar to a predetermined shape of a natural red blood cell. In certain aspects, the predetermined shape of a natural red blood cell is natural and healthy red blood cell. In other aspects, the predetermined shape that is being mimicked by the synthetic red blood cells formed in accordance with the present teachings is a natural, but malformed, red blood cell that contributes to a diseased condition in a mammal, such as a human. As noted above, in alternative variations, the predetermined shape is not limited to that of a red blood cell, but rather may be formed from a template having a shape similar to other cells in an organism, as appreciated by those of skill in the art.

The red blood cell particles of the present teachings can be employed in vitro, for example, used in assays, cell cultures, or the like, or introduced to an organism in vivo, for example, introducing the synthetic red blood cells into a mammal (e.g., a human). Such a synthetic red blood cell can be used in methods to deliver a bioactive agent in the form of a pharmaceutical active agent (e.g., a drug) into an organism, for example, via the organism's circulatory system and therefore in vivo. Thus, by introducing it to a synthetic red blood cell particle having a bioactive agent comprising an active ingredient selected from the group consisting of: a therapeutic active ingredient, a systemic active ingredient, a chemotherapy active ingredient, a localized active ingredient, a nutritional active ingredient, and combinations thereof. In certain aspects, a synthetic red blood cell formed according to the present technology can be used in in vivo or in vitro methods that target delivery of a bioactive agent to a target within an organism by introducing a synthetic red blood cell particle as described above, where the bioactive agent includes an active agent and a targeting moiety for interacting with and delivering the synthetic red blood cell particle to the target. In certain other aspects, methods of diagnosing a disorder in an organism can be achieved by introducing a synthetic red blood cell particle of the present disclosure into a circulatory system of the organism, where the bioactive agent comprises a diagnostic imaging indicator agent. In yet other aspects, the present disclosure provides methods of detoxification of an organism having a toxin, which includes introducing a synthetic red blood cell particle as described herein, where the bioactive agent comprises an agent that interacts with such a toxin to facilitate removal from the organism (for example, by binding and filtration at the liver and/or kidneys) or neutralization of the toxin to prevent harmful effects.

With renewed reference to the methods of forming red blood cell-mimetic particles, in one embodiment, a spherical or elliptical particle comprises a polymer. For example, one suitable polymer is a polystyrene microsphere with a high elastic modulus, which is used as a starting template. See for example, particle 110 FIG. 1A. Conventionally, changing the shape of a solid polystyrene microparticle into a discoid shape like that of a RBC is quite challenging. The present teachings, however, successfully provide methods to treat a polymeric template (e.g., a polystyrene spherical polymer or an electrohydrodynamically jetted polyester particle, like a PLGA disk or sphere) to change the shape and mechanical properties to create a morphology similar to an RBC-like particles (FIGS. 1A-1B). For example, in accordance with the present teachings, hollow polystyrene particles (e.g., 112 in FIG. 1A), especially hollow spheres, upon solvent or heat-induced fluidization, collapse into a desired RBC shape (e.g., 120 in FIG. 1A). By way of example, hollow polystyrene spheres (1 μm diameter, 400 nm shell thickness) can be used as a starting template.

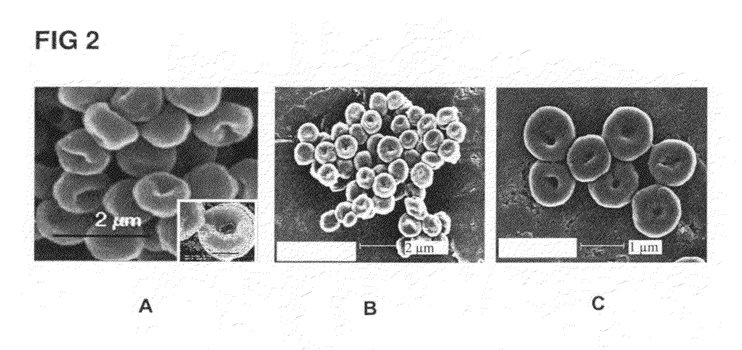

In this exemplary embodiment of the present disclosure, a layer-by-layer (LbL) self assembly technique is utilized to electrostatically deposit bioactive agents that are either cationic or anionic molecules or polymers on a surface of the template particle. For example, materials such as bovine serum albumin (BSA) and poly(allylamine hydrochloride) (PAH) can be selected as the polyanion and polycation to form bioactive layers, respectively. The stepwise (sequential) adsorption of BSA and PAH onto template particle surfaces is generally mediated by hydrophobic and electrostatic interactions. Following the adsorption of multiple (sequential) layers, the shell is cross-linked using glutaraldehyde to provide stability to respective BSA and PAH layers of the particles. The template polymer core can then be exposed to a solvent or other treatment agent, such as tetrahydrofuran (THF), to induce collapse of the template particle and thus to form an RBC-shaped particles (FIG. 2A). While not limiting the disclosure to any particular theory, the template collapse is generally believed to be induced by two factors; fluidization and partial solubilization of the polymer core of the template and the build-up of an osmotic gradient across the shell due to the presence of solvent on the outside and water on the inside of the template shell. In this manner, the template is suitably collapsed/loses rigidity to permit the bioactive shell layers to form a shape of an RBC particle.

In another preferred aspect of the present disclosure, alternate methods are provided to prepare RBC-mimetic particles having a similar morphology to natural red blood cell particles and having one or more bioactive proteins innate to red blood cells, such as hemoglobin (Hb). Hb is the main constituent of natural RBCs and is approximately 92% by dry weight. Hb is a tetramer with each chain non-covalently bound to each other. The protein further carries one heme group, to which oxygen and other small molecules can bind reversibly. In one variation, poly(4-styrene sulfonate) (PSS) and Hb are used as complementary polyelectrolytes for the LbL assembly to yield RBC-shaped particles (FIG. 2B). Alternatively, Hb can be adsorbed onto the surface of the template particles, cross-linked with glutaraldehyde, followed by the dissolution of the core. The morphology of the particles is substantially similar to those of the LbL particles described above (FIG. 2C). The method described here desirably yields soft and synthetic RBC-mimetic particles (referred to herein as “sRBCs”). It should be noted that the synthetic particles formed in accordance with the present teachings may mimic select and/or key features of a natural RBC, but not necessarily all the features of natural RBCs. For example, the synthetic red blood cells of the present disclosure desirably share one or more of the following desirable attributes with a natural red blood cell: size, shape, elastic modulus, ability to deform under flow, oxygen-carrying capacity, and/or carbon dioxide-carrying capacity. In certain aspects, a synthetic red blood cell of the present disclosure has all of these desirable attributes.

In yet other aspects, a variety of biocompatible materials may be used to form a template in the process of forming sRBCs. Moreover, in various aspects, the methods of the present disclosure provide greater control over the size of the template and the resulting SBC-mimetic particles formed. For example, natural RBCs generally have an average diameter of about 7 μm. A particularly suitable biocompatible and biodegradable material for forming a template is poly(lactic acid-co-glycolide) (PLGA). Other non-limiting suitable polymers were discussed previously above. During synthesis of the template and sRBCs particles, the size of PLGA particles can be highly controlled. For example, RBC-shaped templates comprising PLGA optionally have an average particle size of 7±2 μm.

For this purpose, spherical or other shaped template particles of appropriate sizes may be prepared using an electrohydrodynamic jetting process, such as those described in U.S. Pat. No. 7,767,017 to Lahann et al.; and U.S. Publication No. 2007/0237800 (U.S. Ser. No. 11/763,842); and U.S. patent application Ser. No. 12/257,945 filed on Oct. 24, 2008 and entitled “Methods for Forming Biodegradable Nanocomponents With Controlled Shapes and Sized Via Electrified Jetting,” incorporated herein by reference, the various operating regimes for forming nanocomponent particle morphologies are described, including appropriate process parameters for forming sphere, discs and the like, among others.

In FIG. 1B, a “side-by-side” configuration of Fluids A and B 100, 102 are combined to form a pendant droplet 104 of conducting liquid. The two Fluids A and B in FIG. 1 are merely exemplary and non-limiting, as multiple fluids can be jetted to form a plurality of phases, depending on the template particle desired, as described further below. The drop 104 is exposed to an electric potential 142 of a few kilovolts, where the force balance between electric field and surface tension causes the meniscus of the pendent droplet 104.to develop a conical shape, the so-called Taylor cone (not shown). Above a critical point, a highly charged liquid jet is ejected from an apex of the cone.

As schematically presented in FIG. 1B, the biphasic jet that is ejected by the stable biphasic cone is continuous (i.e., not fragmented) and can solidify into biphasic particles. The two phases, i.e., the two jetting liquid streams (or solutions) are optionally compatible with each other (e.g., miscible or soluble) or in certain alternate variations are incompatible. Where the two polymer solutions are compatible with each other, a stable cone-jet forms a stable interface between the two phases. In such situations, it is believed that the process is kinetically controlled (rather than thermodynamically controlled), resulting in one phase being trapped in each side before they mix with the other phase.

Channels 130, 132 are configured adjacent to each other (i.e., side by side) in nozzle 134. As noted above, the setup of the electrified jetting apparatus is exemplary and not limited in number of channels or configuration of the respective channels. It should be noted that a single capillary may be employed to form a single phase of particle in a predetermined shape, although the example employs dual capillaries. A syringe pump (not shown) is used to drive the liquids in nozzle 134. In some variations, channels 130, 132 are capillaries. Channels 130, 132 feed two different jetting liquid streams 136, 138 into region 140 having an electric field generated by power supply 142. Channels 130, 132 are of sufficient dimensions to allow contact of liquids streams 100, 102 to drop 104, which forms composite stream 128. In one variation, this electric field is generated by the potential difference between nozzle 134 and receiving substrate plate 146. Typically, an electric field is formed by applying a potential difference between at least two electrodes from about 0.1 kV to about 25 kV. Various configurations of plates and geometries (electrodes) may be used to generate the electric field as known to those of skill in the art and are contemplated by the present disclosure.

Since the electrified jetting methods are related to electrohydrodynamic processes, the properties of the jetting liquid and operating parameters are interrelated. Moreover, when the jetting liquids are not one-component systems (i.e., mixtures of two or more compounds), the jetting liquid is a solution having properties governed by several parameters of the solvent and solutes. It should be appreciated that liquid properties, solution parameters, and operating parameters are related, as recognized by those of skill in the art. Relevant material properties that affect the shape of particles that are formed include viscosity, surface tension, volatility, thermal and electrical conductivity, dielectric permittivity, and density. Relevant solution properties include polymer concentrations, molecular weight of polymer, solvent mixtures, surfactant(s), doping agent(s), and cross-linking agent(s). Relevant operating parameters include flow rate of the liquid streams, electric potential, temperature, humidity, and ambient pressure. With regard to the operating parameters, the average size and size distributions of the droplets in electrospraying with cone-jet mode is generally dependent on the flow rate (pumping rate of the jetting liquids).

At a fixed flow rate, one or several relatively monodisperse classes of nano-component diameters are formed. At minimum flow rates, the modality of the distributions and diameter of the droplet itself also show their minima. When the flow rate is changed, the electric field can be adjusted by changing either distance or electric potential between the electrodes in order to sustain a stable cone-jet mode. Higher flow rates may be accompanied by a higher electrical field applied for mass balance of jetting liquids.

In certain aspects, the process dependent variables which are used to control particle shape to arrive at a predetermined particle shape, include, but are not limited to, concentration of polymers in and conductivity of the respective jetting solutions, as well as flow rates of the jetting streams. The concentration of a polymer (along with other components) in a solution/jetting stream influences the viscosity, as does the molecular weight of the polymer (and other components, where present). Solvents or vehicles used in the jetting solution impact the dielectric constant of a respective jetting stream, viscosity, and vapor pressure. The flow rate of the jetting liquid stream relates to vapor pressure and stability of the jet formed. In certain aspects, the distance between a collector and a needle tip impacts the strength of the electric field applied, which in turn can impact the stability of the cone, as well as the cone shape itself and thus voltage, formed during jetting. Generally, so long as a stable cone jet is formed via correct distance between the electrode and the nozzle/needle tip, this variable does not have a significant impact on nano-component particle shape. Temperature, pressure, and humidity likewise impact the behavior of the jetting fluids and shapes formed, impacting solvent volatilization and applied voltage, for example.

The set of capillaries 130, 132 is arranged in a side-by-side configuration. Under these conditions, a well-defined interface can be formed within the pendant droplet. Upon application of a sufficiently high threshold voltage, accumulation of surface charges results in the formation of a liquid cone. The liquid cone acts as the origin of a polymer jet that retains the multiphasic geometry of the initial droplet through jet elongation, solvent evaporation, and polymer solidification. In these multiphasic particles, individual phases or compartments can differ with respect to their chemical compositions, which can be controlled by controlling the composition of the initial jetting solutions. Thus, individual phases may be comprised of a variety of different additives, such as functional polymers, dyes, biomolecules, and/or active agents.

As further described in co-pending parent U.S. patent application Ser. No. 12/257,945 filed on Oct. 24, 2008 and entitled “Methods for Forming Biodegradable Nanocomponents With Controlled Shapes and Sized Via Electrified Jetting,” incorporated herein by reference, the various operating regimes for forming particle morphologies are described, including appropriate process parameters for forming spheres and disc shapes, among others. Where polymer concentration is lower and flow rates remain relatively low, discs are formed. If flow rate is lower or intermediate for a relatively high concentration spheres are formed. When relatively high flow rates are employed during electrojetting with an intermediate polymer concentration, rod or cylinder shapes are formed.

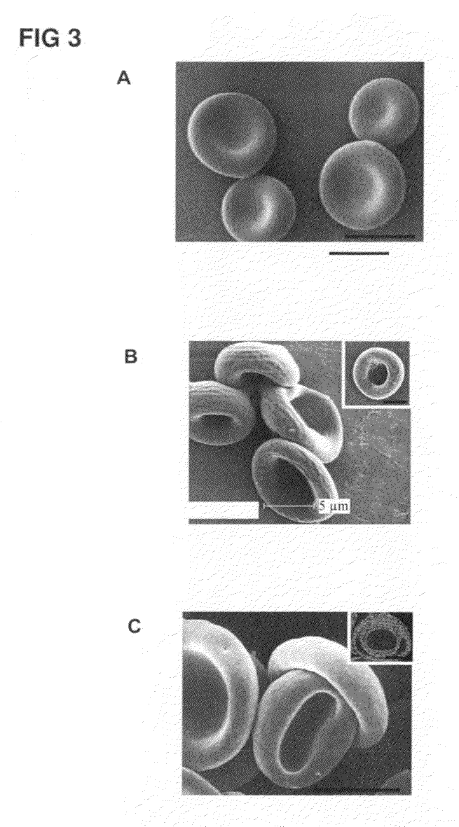

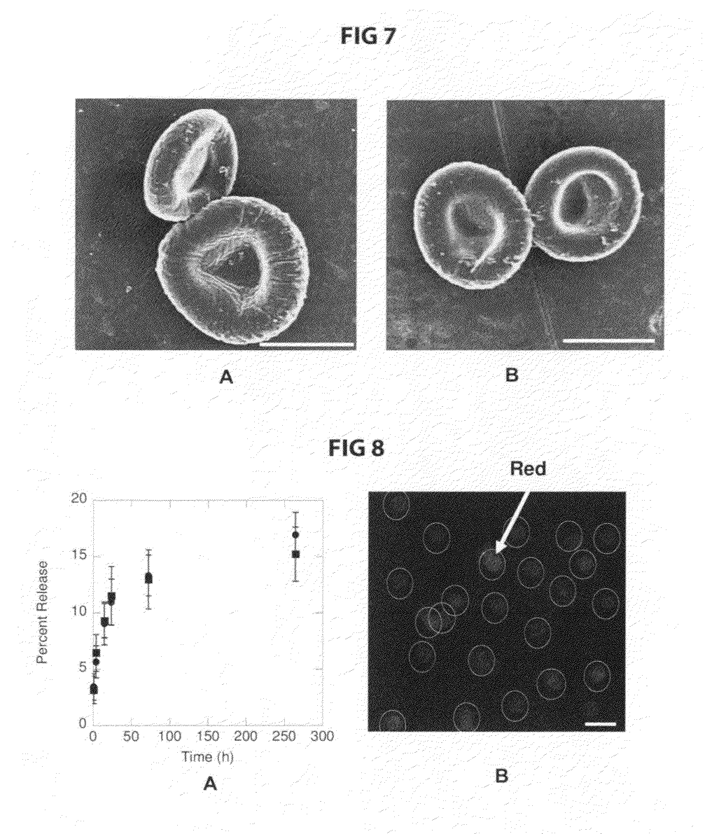

After formation via either the techniques of FIG. 1A or 1B, the template particles 112 or 150 having a shell material (with one or more bioactive layers) created thereon with optional cross-linking (154 in FIG. 1B or 122 in FIG. 1A), the particles (with both shell and template particle intact) are then incubated in a solvent or treatment agent, such as 2-propanol, to induce formation of an RBC-shaped PLGA template particle (FIG. 3A). As discussed above, other techniques for inducing collapse, such as heat fluidization, are likewise contemplated. A range of different template particle sizes can be prepared via this technique, including smaller template particles (e.g., 3±1.5 μm). Such template particles yield soft, protein-based biocompatible particles using the modified LbL technique according to the present teachings. For example, in one embodiment shown in FIG. 1B, nine alternate layers of either Hb/BSA or PAH/BSA are assembled on the templates 152. The layers are then cross-linked and the underlying PLGA core can be removed using a mixture of 2-propanol and THF (see 160 in FIG. 1B). For example, a treatment mixture having a ratio of 1:2 of 2-propanol to THF can be used to remove the PLGA template core to form sRBCs (FIG. 3B, PAH/BSA sRBCs or FIGS. 7A-7C for images of sRBCs made from LbL Hb/BSA). In certain aspects, the ratio of 2-propanol to THF avoids incomplete dissolution of the PLGA core in the template (by use of excess 2-propanol) and complete collapse (by use of excess THF). sRBCs synthesized by such a method demonstrate close resemblance to natural RBCs (FIGS. 3A-3B; 10A-10C b-sRBCs, c-mouse RBCs).

In various aspects, the present disclosure provides a method of making a red blood cell-mimetic particle comprising the steps of providing a template particle comprising a polymer. The polymer of the template is preferably comprises a pharmaceutically and/or cosmetically acceptable polymer. In certain preferred aspects, the polymer of the template comprises poly(lactide-co-glycolide polymer (PLGA) or polystyrene (PS). At least one bioactive agent is then disposed on a surface of the template particle to form a stable bioactive layer. Next, the template particle is substantially removed with a treatment agent, so that the stable bioactive layer forms a particle having a substantially similar shape to a predetermined shape of a natural red blood cell. The treatment agent optionally comprises 2-propanol, tetrahydrofuran, or combinations thereof. In certain embodiments, the treatment agent comprises a ratio of 2-propanol to tetrahydrofuran is about 1:2 on a weight basis.

In certain variations, the methods of the present disclosure further comprise incubating the template particle having a first shape in the presence of an agent that induces the template particle to have a second shape distinct from the first shape, wherein the second shape is a substantially similar shape to the predetermined shape of the natural red blood cell. In various aspects, the first shape is optionally a sphere and the second shape is a biconcave discoid shape (like in FIG. 1C). In certain alternate embodiments, the predetermined shape of a natural red blood cell is a malformed red blood cell contributing to a diseased condition in a mammal.

The method may further comprise forming the template particle by electrohydrodynamic jetting, which comprises jetting a liquid stream passing through an electric field generated by electrodes sufficient to form a cone jet. In certain variations, the template particle formed via electrohydrodynamic jetting has a discoid shape. In other aspects, the template particle formed via electrohydrodynamic jetting has a first shape selected from the group consisting of: a substantially spherical, a substantially ellipsoidal, and a substantially discoid shape, and the providing step further comprises incubating the template particle having the first shape in the presence of an agent that induces the template particle to have a second bi-concave discoid shape. The methods of the present disclosure optionally provide a step of disposing of the bioactive agent via a layer-by-layer (LbL) self-assembly process. The bioactive agent may further comprise cross-linking the bioactive agent on the surface of the template to form the stable bioactive layer. The bioactive agent may optionally comprise an active ingredient selected from the group consisting of: a therapeutic active ingredient, a systemic active ingredient, a chemotherapy active ingredient, a localized active ingredient, a nutritional active ingredient, a diagnostic imaging indicator agent, and combinations thereof. In certain preferred aspects, the bioactive agent optionally comprises an ingredient selected from the group consisting of: bovine serum albumin (BSA), poly(allylamine hydrochloride) (PAH), hemoglobin (Hb), and combinations thereof.

In yet other aspects, the present teachings provide a synthetic red blood cell particle comprising a stable bioactive layer comprising at least one bioactive agent and having a bi-concave discoid shape substantially similar to a predetermined shape of a natural red blood cell. In various aspects, the bioactive agent comprises an active ingredient selected from the group consisting of: a therapeutic active ingredient, a systemic active ingredient, a chemotherapy active ingredient, a localized active ingredient, a nutritional active ingredient, a diagnostic imaging indicator agent, or any agent described above, including combinations thereof. In certain preferred variations, the bioactive agent is selected from the group consisting of: bovine serum albumin (BSA), poly(allylamine hydrochloride) (PAH), hemoglobin (Hb), and combinations thereof.

The present disclosure also provides a method of delivering a drug within an organism by introducing a synthetic red blood cell particle, like those described above, into the organism. In other variations, the present disclosure provides a method of delivering a drug to a target within an organism by introducing a synthetic red blood cell particle (as described above) into the organism, where the bioactive agent includes an active agent and a targeting moiety for interacting with and delivering the synthetic red blood cell particle to the target.

In other aspects, a method of diagnosing a disorder in an organism is provided by introducing a synthetic red blood cell particle (as described above) into the organism, where the bioactive agent comprises a diagnostic imaging indicator agent. The present disclosure further provides a method of transporting oxygen in an organism by introducing the inventive synthetic red blood cell particles into a circulatory system of the organism, where the bioactive agent comprises an oxygen-releasing component, such as hemoglobin that release oxygen. Likewise, a method of transporting carbon dioxide is provided in certain variations in an organism that includes introducing an inventive synthetic red blood cell particle into a circulatory system of the organism, where the bioactive agent transports carbon dioxide. Such a bioactive agent may be hemoglobin (which also can transport carbon monoxide, for example in the case of carbon monoxide poisoning). As appreciated by those of skill in the art, the same bioactive agent (e.g., hemoglobin) can be used in an organism to both release oxygen and transport carbon dioxide, mimicking the function of natural red blood cells in an organism.

Further, the present disclosure provides for methods of detoxification for an organism having a toxin by introducing an inventive synthetic red blood cell particle into the organism. The bioactive agent comprises an agent that interacts with one or more toxins to facilitate removal from the organism or neutralization of the toxin(s).

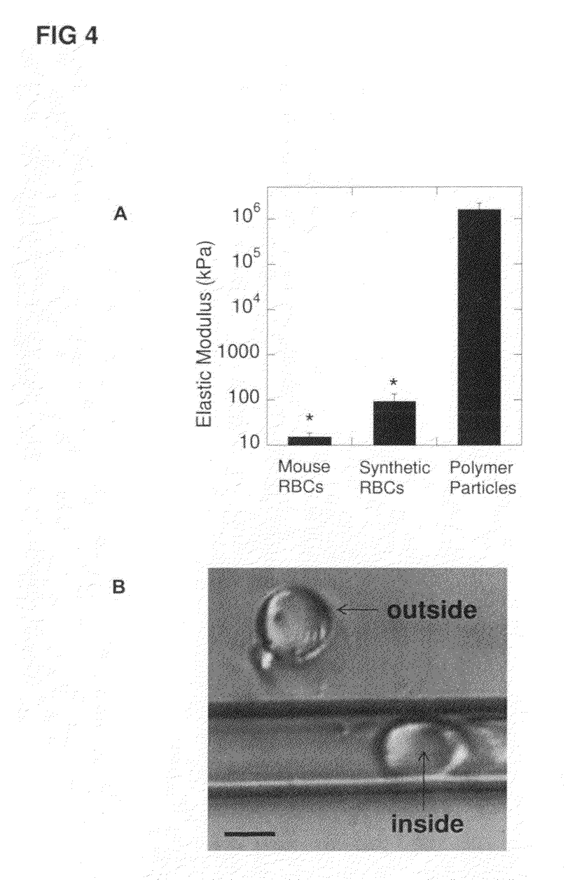

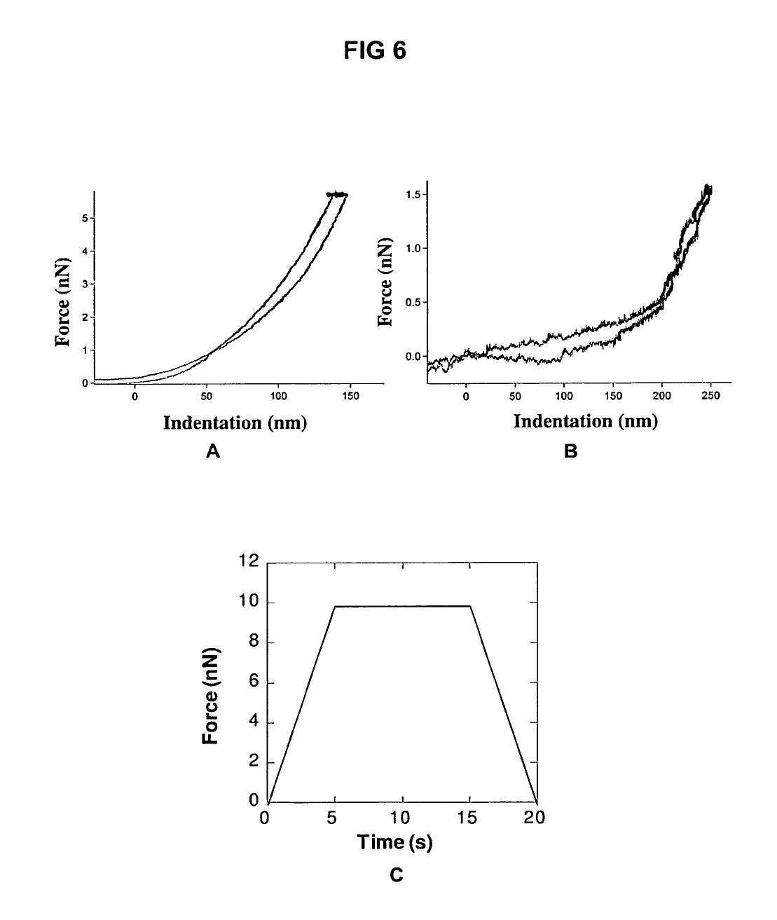

sRBCs formed in accordance with these principles are flexible, owing to the dissolution of the template PLGA core which leaves behind a soft protein shell (FIG. 4A). The elastic modulus of sRBCs can be measured using Atomic Force Microscopy (AFM). AFM has been previously used to measure elastic modulus of soft materials such as LbL films, hollow protein particles and platelet. A wide range of elastic moduli have been reported for LbL structures in the range of 10 kPa to >100 MPa depending on several parameters including the template/shell materials, shell density, shell crosslinking and pH, among many others. The elastic modulus of sRBCs can be obtained from force-indentation curves obtained by inducing deformations comparable to the capsule wall thickness, where the elastic response is expected. The typical loading-unloading cycle used for this study and the corresponding force curves obtained for sRBCs (FIGS. 6A-6C). The elastic modulus of sRBCs (92.8±42 kPa) is found to be four orders of magnitude lower than that of PLGA template particles (1.6±0.6 GPa) and of the same order of magnitude as that of natural RBCs. The elastic modulus of mouse RBCs is found to be 15.2 kPa±3.5 kPa, which is consistent with the values reported in literature. The data in FIG. 4A clearly indicates that sRBCs approximate or mimic natural RBCs behavior with respect to mechanical properties.

The flexibility of sRBCs (7±2 μm) is confirmed by flowing them through narrow glass capillaries (5 μm inner diameter) and visualizing the stretching (FIG. 4B, two sRBCs, one inside the capillary and one outside the capillary). Whereas the particle outside the capillary is symmetric and circular, the particle inside the capillary is stretched due to flow (FIG. 4B). The average aspect ratio of stretching is found to be 170±25.8% (n=22). Further, particles are able to regain their discoidal shape upon exiting the capillary, confirming the reversible nature of the shape deformation. Thus, similar to their natural counterpart, sRBCs maintain the ability to flow through channels smaller than their resting diameter and stretch in response to flow.

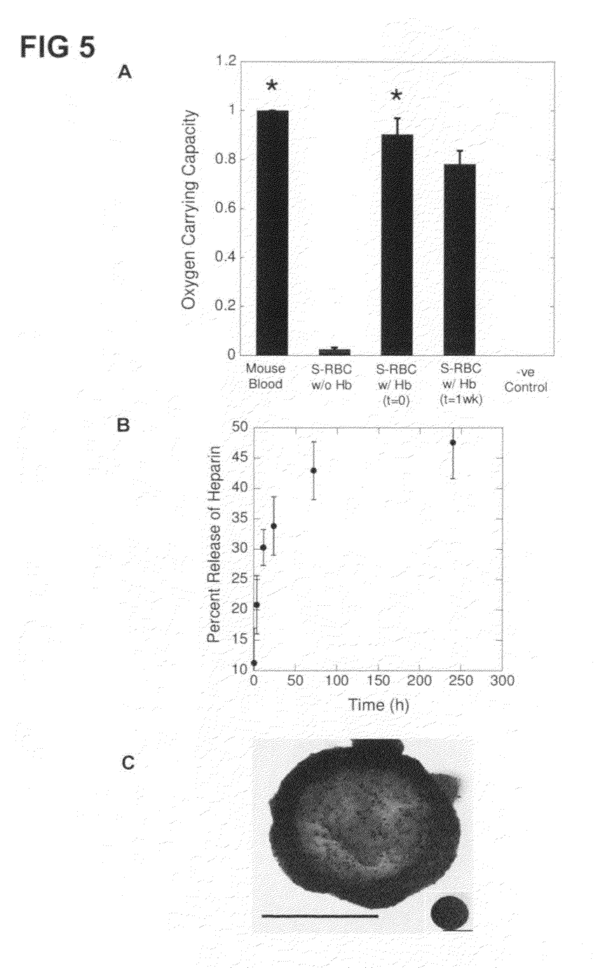

sRBCs have numerous biomedical applications, including in vivo and in vitro uses. Since the primary function of natural RBCs is to deliver oxygen to the various tissues of the body (and optionally to remove carbon dioxide or carbon monoxide), the ability of sRBCs to bind oxygen is depicted in FIG. 5A, for example. In certain variations, cross-linking and exposure to solvent during particle preparation may potentially lead to deactivation of Hb, thereby limiting its oxygen and/or carbon dioxide carrying capacities (FIG. 5A, sRBC without Hb). To enhance oxygen-carrying capacity of sRBCs, particles are optionally further fortified in certain aspects with additional, uncrosslinked bioactive material, such as Hb. This procedure results in high oxygen binding levels (FIG. 5A, sRBC with Hb, time=0) compared to the positive control, which is mouse blood. About 90% of this oxygen carrying capacity is retained even after one week (FIG. 5A, sRBC with Hb, time=1 week). Included is a negative control, BSA coated particles, which showed no ability to bind oxygen (FIG. 5A, negative control).

Therefore, sRBCs formed in accordance with the principles of the present disclosure are excellent for delivery of bioactive agents, like drugs, especially in the vascular compartment. In certain variations, these sRBC particles can be loaded with drugs by incubation in solutions containing the drug. By way of example, a model molecule, Texas Red conjugated dextran (3 kDa and 10 kDa molecular weight) is loaded into the sRBCs by direct incubation. FIGS. 8A-8B. Both molecules penetrate into the interior of the sRBCs. Dextran is subsequently released from these particles in a controlled manner.

Once the release of dextran is confirmed, controlled release of a therapeutic drug heparin (10-15 kDa) is tested. Heparin is widely used as an anti-coagulant for the treatment of thrombosis. Parenteral administration of heparin can result in severe side effects such as heparin-induced thrombocytopenia, elevation of serum aminotransferase levels, hyperkalemia, alopecia, and osteoporosis. The sRBCs show high amounts of heparin loading (70 μg of heparin per mg of particles) and continuous release over a period of several days in vitro (FIG. 5B).

The sRBCs of the present technology can also be used for medical imaging applications. For example, iron oxide nanocrystals with an average diameter of 30 nm are encapsulated inside the PLGA RBC-shaped particles prepared via electrohydrodynamic jetting. Incorporation of iron oxide nanoparticles makes particles suitable as contrast agents for Magnetic Resonance Imaging (MRI). An important requirement for this use is homogenous dispersion of the iron oxide nanocrystals. As shown in FIG. 5C, Transmission Electron Microscopy (TEM) images show well-distributed iron oxide particles in the PLGA matrix. The inset shows TEM image of a spherical PLGA particle prior to shape modification. Magnetic particles are currently being developed for a wide spectrum of applications such as MRI contrast agents for diseases such as atherosclerotic plaque, targeted therapeutic delivery, hyperthermia treatment for cancerous tumors. The interior of the particles described here can be further engineered by the formation of separate compartments using electrohydrodynamic co-jetting process (FIG. 1B) to incorporate one or more agents of interest.

At the same time, the surface can be engineered by adsorption of additional proteins such as CD47, a ubiquitous self-marker expressed on the surface of RBCs. The particle surface can also be modified with hydrophilic polymers, such as PEG, depending on the application for which the RBC will be used.

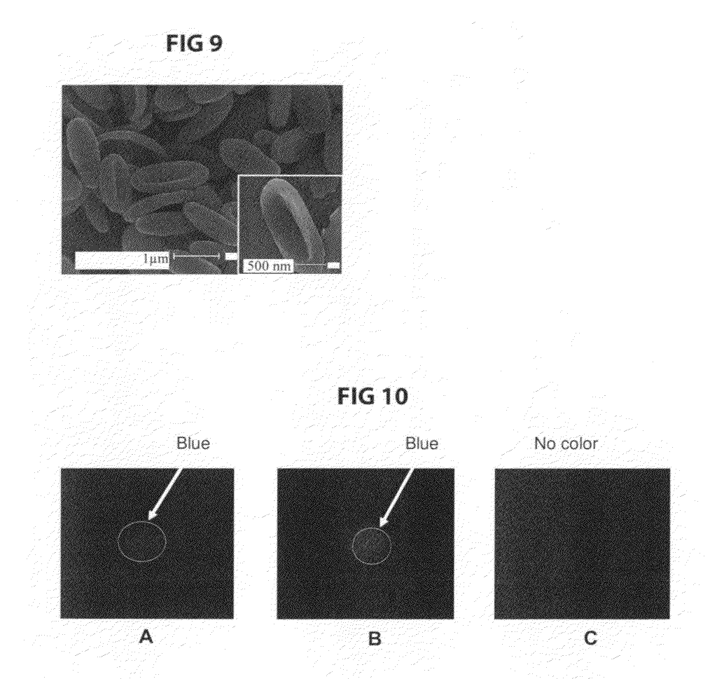

In addition to preparing particles that mimic the shape and properties of healthy RBCs, the present teachings can also be used to design particles that mimic the shape and properties of diseased cells. For example, hereditary elliptocytosis is a disease that leads to the formation of elliptical RBCs, a shape is mimicked in our method (see FIG. 9). Other examples of diseased conditions where the shape of RBCs is altered include spherocytosis and sickle-cell anemia. Such disease cell mimicking particles can serve as synthetic models to help elucidate the effect of transformation in physical properties of RBCs in these disease conditions.

Drug delivery carriers, which mimic the structural and functional properties of RBCs, have the potential to address some of the key challenges faced by current drug delivery carriers. The methods of the present disclosure provide synthetic particles that mimicry of many key attributes of natural RBCs, including the size, shape, elastic modulus, ability to deform under flow and oxygen-carrying capacity. In addition, it is contemplated that incorporation of additional functionalities such as therapeutic and diagnostic agents in these carriers, provide a vast array of applicability for drug delivery, medical imaging, detoxification, and the establishment of new, improved disease models.

The materials employed in the following examples are PSS polymer (MW˜70 kDa), PAH (Mw˜50 kDa), BSA, human Hb, PLGA with a lactide:glycolide ratio of 85:15 (MW=40-75 kDa), chloroform, N,N-dimethylformamide, heparin, luminol, sodium perborate, sodium carbonate, 2-propanol, toluene, phosphate buffered saline (PBS) tablets, sodium citrate, poly(vinyl alcohol) (PVA fully hydrolyzed) are obtained from Sigma Aldrich (St. Louis, Mo., USA). Polybead hollow microspheres (5.21% solids, 1 μm in diameter) are commercially available from Polysciences (Warrington, Pa., USA). Texas Red conjugated dextran (MW 3 kDa, 10 kDa) and anti-fade agent are commercially available from Invitrogen (Carlsbad, Calif., USA). THF, mineral oil, and glycerol are commercially available from EMD Biosciences (San Diego, Calif., USA). Solvable is commercially available from Perkin Elmer (Waltham, Mass., USA). Dialysis cassettes (MWCO=2500) are obtained from Thermo Scientific (Hudson, N.H., USA). 1 mL syringes are used from BD (Franklin Lakes, N.J., USA) and 23 gauge, 1.5 inch long single capillary stainless steel tip is from EFD (East Providence, R.I., USA). Iron oxide nanoparticles of 30 nm diameter suspended in chloroform with oleic acid stabilization are commercially available from Ocean Nanotech (Springdale, Ark., USA). 5-8 μm filters are sold by Millipore (Billerica, Mass., USA).

In one example, the RBC-mimicking particles are formed by the following process. Layer by layer (LbL) assembly is employed to electrostatically adsorb proteins or polyelectrolytes (PEs) on the surface of hollow polystyrene particles. Proteins or PEs are incubated with template particles at a concentration of 2 mg/mL in 0.5 M NaCl solution for 20 minutes on a shaker plate at 350 rpm, followed by three washings (centrifugation and re-suspension) in 0.5 M NaCl. For example, stepwise shell formation composed of BSA and polycation PAH is performed until four bilayers are deposited onto the polybead hollow microparticles (108 particles/mL) (BSA/PAH)4. Alternate layers of Hb and PSS are also used to construct the shell of RBC-mimicking particles.

Next, the layers are cross-linked using the following procedure. 500 μl of a 2.5% glutaraldehyde solution in 0.2 M sodium cacodylate buffer is added to protein coated microparticles and left to incubate on a shaker plate for one hour. Next, the particles are sonicated and a stop solution of 30 mM sodium borohydride is added to the particle solution for 30 minutes followed by 3 wash steps with 0.01 M PBS. The particle solution is placed in a dialysis cassette in 0.01 M PBS. After the first hour in dialysis, 400 mL of fresh 0.01 M PBS is added to the reservoir. After 24 hours in the dialysis cassette, the particle solution is removed and centrifuged. In order to dissolve the polymeric core, cross-linked particles are incubated with THF, vortexed and then sonicated. Template polymeric particles are dissolved in THF for approximately 12 hours. Polybead oligomers are removed by washing with 1 mL of THF two times (vortexed and centrifuged). Particles are then washed four times with 1 mL of 0.5 M NaCl followed by overnight dialysis in 0.5 M NaCl. Particles are finally re-suspended in either 0.5M NaCl or PBS or deionized water to ensure complete removal of the solvent from the particles.

sRBCs are also prepared by deposition of only Hb layers by incubating the particles with 2 mg/mL Hb for 4 hours on a shaker plate followed by cross-linking using 5% glutaraldehyde as mentioned above. The polymeric template is then dissolved using THF. For chemiluminescence experiments, sRBCs are fortified with Hb by incubation with Hb solution (2 mg/mL) for one hour. For sustained oxygen carrying capacity experiments, sRBCs fortified with Hb are washed three times with PBS and incubated in PBS for 7 days. The particles are washed again with PBS and then used to determine oxygen carrying capacity.

A similar procedure is adopted for the fabrication of particles from PLGA template particles. Nine alternate layers of Hb/BSA or PAH/BSA are deposited and the layers are cross-linked using glutaraldehyde. A mixture of THF and 2-propanol of varying concentrations (10:1, 5:1. 2:1 and 1:1) is used to dissolve the template PLGA particles.

Synthesis of PLGA particles occurs as follows. The experimental setup used for electrohydrodynamic jetting is described above. Briefly, a 4.5% (w/w) solution of PLGA in 97:3 (by volume) CHCl₃: DMF is drawn in a syringe and pumped at 0.7 mL/h via a syringe pump (KDS100, KD Scientific, Massachusetts, USA). A single capillary is connected to the tip of the syringe and further attached to the cathode of a high-voltage supply (Gamma High Voltage Source, USA). The voltage is controlled in the range of 5.7-6 kV. A square piece of aluminum foil is used as the anode, which also acted as a collecting substrate. The distance between the electrodes is maintained in the range of 25-30 cm.

PLGA RBC-shaped template particles are formed from substantially spherical particles obtained by electrohydrodynamic jetting. The substantially spherical particles are harvested from the substrate and incubated for 12 hours in 2-propanol at room temperature (1 mL of 2-propanol per 2 mg of particles). The particles are then centrifuged and re-suspended in DI water containing 0.01% Tween-20. Alternatively, the use of higher flow rates in the electrohydrodynamic jetting results in preparation of particles that collapse into red-blood cell shaped templates, without incubation in a solvent agent.

Iron oxide nanoparticle encapsulation in PLGA RBC-shaped particles occurs as follows. For encapsulation of iron oxide nanoparticles into the PLGA particles, electrohydrodynamic jetting is carried out using a 3.8 wt. PLGA in 95:5 CHCl3: DMF (by vol.), and 30 nm iron oxide nanoparticles with oleic acid surface stabilization are added at about 12% by weight of total PLGA. Flow rates from 0.08-0.1 mL/h and voltages in the range of 3.9-4.5 kV are employed.

Mouse red blood cells are harvested as follows. Mouse blood is obtained by cardiac puncture, collected in heparinized tubes and diluted in 4% sodium citrate buffer (pH 7.4). The red blood cells are isolated by centrifugation at 100 g for 3 minutes. These are then used for the chemiluminescence experiments in appropriate concentrations. For scanning electron microscopy, the cells are cross-linked using 2% glutaraldehyde for 2 hours and washed with sodium citrate buffer.

The foregoing description of the embodiments has been provided for purposes of illustration and description. It is not intended to be exhaustive or to limit the invention. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the invention, and all such modifications are intended to be included within the scope of the invention.

The following attachments, designated Appendices A and B, are incorporated by reference herein in their entirety and more particularly describe certain features and aspects of various embodiments of the present teachings.

1. A method of making a red blood cell-mimetic particle comprising:
providing a template particle comprising a polymer;
disposing at least one bioactive agent on a surface of said template particle to form a stable bioactive layer; and
substantially removing said template particle with a treatment agent so that said stable bioactive layer forms a particle having a substantially similar shape to a predetermined shape of a natural red blood cell.

2. The method of claim 1, wherein said providing of said template particle further comprises incubating said template particle having a first shape in the presence of an agent that induces said template particle to have a second shape distinct from said first shape, wherein said second shape is a substantially similar shape to said predetermined shape of said natural red blood cell.

3. The method of claim 2, wherein said first shape is a sphere and said second shape is a biconcave discoid shape.

4. The method of claim 1, wherein said providing further comprises forming said template particle by electrohydrodynamic jetting that comprises jetting a liquid stream passing through an electric field generated by electrodes sufficient to form a cone jet.

5. The method of claim 4, wherein said template particle formed via electrohydrodynamic jetting has a discoid shape.

6. The method of claim 4, wherein said template particle formed via electrohydrodynamic jetting has a first shape selected from the group consisting of: a substantially spherical, a substantially ellipsoidal, and a substantially discoid shape, and said providing further comprises incubating said template particle having said first shape in the presence of an agent that induces said template particle to have a second biconcave discoid shape.

7. The method of claim 1, wherein said disposing of said bioactive agent is conducted via a layer-by-layer (LbL) self-assembly process.

8. The method of claim 1, wherein said disposing of said bioactive agent further comprises cross-linking said bioactive agent on said surface of said template to form said stable bioactive layer.

9. The method of claim 1, wherein said bioactive agent comprises an ingredient selected from the group consisting of: bovine serum albumin (BSA), poly(allylamine hydrochloride) (PAH), hemoglobin (Hb), and combinations thereof.

10. The method of claim 1, wherein said bioactive agent comprises an active ingredient selected from the group consisting of: a therapeutic active ingredient, a systemic active ingredient, a chemotherapy active ingredient, a localized active ingredient, a nutritional active ingredient, a diagnostic imaging indicator agent, and combinations thereof.

11. The method of claim 1, wherein said polymer of said template is a pharmaceutically and/or cosmetically acceptable polymer.

12. The method of claim 1, wherein said polymer of said template comprises poly(lactide-co-glycolide polymer (PLGA).

13. The method of claim 1, wherein said treatment agent comprises a mixture of 2-propanol and tetrahydrofuran.

14. The method of claim 13, wherein a ratio of 2-propanol to tetrahydrofuran is about 1:2 on a weight basis.

15. The method of claim 1, wherein said predetermined shape of a natural red blood cell is a malformed red blood cell contributing to a diseased condition in a mammal.

16. A synthetic red blood cell particle comprising a stable bioactive layer comprising at least one bioactive agent and having a biconcave discoid shape substantially similar to a predetermined shape of a natural red blood cell.

17. The synthetic red blood cell particle of claim 16, wherein said bioactive agent is selected from the group consisting of: bovine serum albumin (BSA), poly(allylamine hydrochloride) (PAH), hemoglobin (Hb), and combinations thereof.

18. The synthetic red blood cell particle of claim 16, wherein said bioactive agent comprises an active ingredient selected from the group consisting of: a therapeutic active ingredient, a systemic active ingredient, a chemotherapy active ingredient, a localized active ingredient, a nutritional active ingredient, a diagnostic imaging indicator agent, and combinations thereof.

19. A method of delivering a drug within an organism by introducing a synthetic red blood cell particle of claim 16 into the organism, wherein said bioactive agent comprises an active ingredient selected from the group consisting of: a therapeutic active ingredient, a systemic active ingredient, a chemotherapy active ingredient, a localized active ingredient, a nutritional active ingredient, and combinations thereof.

20. A method of delivering a drug to a target within an organism by introducing a synthetic red blood cell particle of claim 16 into the organism, wherein said bioactive agent includes an active agent and a targeting moiety for interacting with and delivering said synthetic red blood cell particle to said target.

21. A method of diagnosing a disorder in an organism by introducing a synthetic red blood cell particle of claim 16 into the organism, wherein said bioactive agent comprises a diagnostic imaging indicator agent.

22. A method of transporting oxygen in an organism by introducing a synthetic red blood cell particle of claim 16 into a circulatory system of the organism, wherein said bioactive agent comprises an oxygen-releasing component.

23. A method of transporting carbon dioxide in an organism by introducing a synthetic red blood cell particle of claim 16 into a circulatory system of the organism, wherein said bioactive agent transports carbon dioxide.

24. A method of detoxification for an organism having a toxin by introducing a synthetic red blood cell particle of claim 16 into the organism, wherein said bioactive agent comprises an agent that interacts with said toxin to facilitate removal from the organism or neutralization of said toxin.
